(19) United States
(12) Patent Application Publication
(10) Pub. No.: US 2023/0079001 A1
(43) Pub. Date: Mar. 16, 2023

(54) SENSOR ASSEMBLY

(71) Applicant: Analog Devices International Unlimited Company, Limerick (IE)

(72) Inventors: Christophe ANTOINE, Newbury (GB); Helen BERNEY, Pennywell (IE); Youri V. PONOMAREV, Rotselaar (BE); Joyce WU, Boston, MA (US)

(73) Assignee: Analog Devices International Unlimited Company, Limerick (IE)

(21) Appl. No.: 17/903,274

(22) Filed: Sep. 6, 2022

Related U.S. Application Data
(60) Provisional application No. 63/242,977, filed on Sep. 10, 2021.

(51) Int. Cl. G01N 27/327

(52) U.S. Cl. CPC G01N 27/3273; G01N 27/3272

(57) ABSTRACT

A sensor assembly for sensing an analyte in a sample matrix comprises an electrode assembly comprising a set of at least one test electrode and may also comprise one or more control electrodes and/or an applicator assembly. The electrode assembly is configured or configurable to define one or more active test electrodes of the set of one or more test electrodes, and at least one of the electrode assembly and the applicator assembly is or are configured or configurable to adjust a quantity of the analyte provided to the active electrode(s), per unit time, for said interaction based at least in part on an analyte characteristic. Alternatively or additionally, the electrode assembly is configured and arranged in a flow path such that the amounts of sample matrix provided to the test electrode(s) and control electrode(s) of the electrode assembly are substantially equal.

### CROSS REFERENCE TO RELATED APPLICATIONS

This application is related to, and claims the benefit of priority from, U.S. Patent Application No. 63/242,977, filed Sep. 10, 2021, titled “SENSOR ASSEMBLY,” the disclosure of which is hereby incorporated by reference herein in its entirety.

### TECHNICAL FIELD OF THE DISCLOSURE

This disclosure relates to a sensor assembly, for example a biosensor or chemical assay, for sensing an analyte in a sample matrix, and a system and method for determining a property of an analyte in a sample matrix.

### BACKGROUND

Various biosensor and chemical assay designs are known for sensing analytes. Analytes may, for example, include biomarkers, such as hormones, established to assist in patient monitoring and/or diagnosis.

In, for instance, standard enzyme-linked immunosorbent assays (ELISA), employed for quantifying analytes such as peptides, proteins, antibodies, and hormones, a recognition element for selectively interacting with, for example binding, the analyte of interest is immobilized on a suitable support. For example, an antigen is immobilized on the support and then complexed with an antibody that is linked to an enzyme.

In biosensors and assays, such as ELISA, it has been found to be challenging to make quantitative measurements of analytes over a wide range of concentrations. Typically, there is a tradeoff between sensitivity and the range of concentrations that can be detected.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1A provides a schematic plan view of a sensor assembly according to some embodiments of the present disclosure;

FIG. 1B provides a schematic plan view of an electrode assembly according to some embodiments of the present disclosure;

FIG. 2 provides a schematic plan view of an electrode assembly according to some embodiments of the present disclosure;

FIG. 3 provides a schematic plan view of an electrode assembly according to some embodiments of the present disclosure;

FIG. 4 provides a schematic plan view of an electrode assembly according to some embodiments of the present disclosure;

FIG. 5 provides a schematic plan view of an electrode assembly according to some embodiments of the present disclosure;

FIG. 6 provides a schematic perspective view of a sensor assembly according to some embodiments of the present disclosure;

FIG. 7 provides a schematic perspective view of a sensor assembly according to some embodiments of the present disclosure;

FIG. 8 provides a schematic plan view of an electrode assembly according to some embodiments of the present disclosure;

FIG. 9 provides a schematic plan view of an electrode assembly according to some embodiments of the present disclosure; and

FIG. 10 provides a flowchart of a method for determining a concentration of an analyte in a sample matrix according to some embodiments of the present disclosure.

### DESCRIPTION OF EXAMPLE EMBODIMENTS OF THE DISCLOSURE

The systems, methods and devices of this disclosure each have several innovative aspects, no single one of which is solely responsible for all of the desirable attributes disclosed herein. Details of one or more implementations of the subject matter described in this specification are set forth in the description below and the accompanying drawings.

Various analyte sensing techniques are known. However, quantitative measurement of analytes over a wide range of concentrations is a challenge. Typically, there is a tradeoff between sensitivity and the range of concentrations that can be detected.

The present disclosure provides a sensor assembly for sensing an analyte. The sensor assembly includes an electrode assembly including at least one active test electrode and may include an applicator assembly. At least one of the electrode assembly and the applicator assembly is or are configured or configurable to adjust a quantity of the analyte provided to the active electrode(s), per unit time, for said interaction based at least in part on an analyte characteristic. Alternatively or additionally, the electrode assembly is configured and arranged in a flow path such that the amount of sample matrix provided to the electrode assembly is substantially equal or equal.

In certain embodiments, a sensor assembly is provided for sensing an analyte in a sample matrix. The sensor assembly includes an electrode assembly including a set of one or more test electrodes configured to interact with the analyte and provide a sensor signal based on said interaction, and an applicator assembly configured to enable application of the sample matrix to the set of one or more test electrodes. The electrode assembly is configured or configurable to define one or more active test electrodes of the set of one or more test electrodes, which one or more active test electrodes contribute to the sensor signal. At least one of the electrode assembly and the applicator assembly is or are configured or configurable to adjust a quantity of the analyte provided to the one or more active test electrodes, per unit time, for said interaction based at least in part on an analyte characteristic.

In certain embodiments, a sensor assembly for sensing an analyte in a sample matrix is provided. The sensor assembly includes an electrode assembly including a set of one or more test electrodes configured to interact with the analyte and provide a sensor signal based on said interaction. The electrode assembly is configured or configurable to define one or more active test electrodes of the set of one or more test electrodes, which one or more active test electrodes contribute to the sensor signal. The electrode assembly is configured or configurable to adjust a quantity of the analyte provided to the one or more active test electrodes, per unit time, for said interaction based at least in part on an analyte characteristic by varying an effective active test electrode area.

In certain embodiments, a sensor assembly for sensing an analyte in a sample matrix is provided. The sensor assembly includes an electrode assembly including a set of one or more test electrodes configured to interact with the analyte and provide a sensor signal based on said interaction and an applicator assembly configured to provide the sample matrix to the set of one or more test electrodes. The applicator assembly is configured or configurable to adjust a quantity of the analyte provided to the one or more test electrodes, per unit time, for said interaction based at least in part on an analyte characteristic by adjusting the amount of the sample matrix provided to the set of one or more test electrodes by the applicator assembly.

In certain embodiments, a sensor assembly for sensing an analyte in a sample matrix is provided. The sensor assembly includes an electrode assembly including a set of one or more test electrodes, the set of one or more test electrodes including an analyte interaction portion configured to interact with the analyte and provide a sensor signal based on said interaction; and a set of one or more control electrodes, the set of one or more control electrodes providing a control electrode area configured for providing a control measurement which is independent of the analyte. The sensor assembly further includes a flow path configured to provide the sample matrix to the electrode assembly. The electrode assembly is configured and arranged in the flow path such that the amount of sample matrix provided to each of the set or one or more test electrodes and the set of one or more control electrodes is substantially equal or equal.

In certain embodiments, a method for determining a property of an analyte in a sample matrix is provided. The method includes: providing the sensor assembly of the preceding embodiment; processing signals received from the electrode assembly including a set of one or more test electrodes configured to interact with the analyte; and determining the property of the analyte in the sample matrix, based at least in part on the sensor signals processed from the electrode assembly, determine the property of the analyte in the sample matrix.

In certain embodiments, a system for determining a property of an analyte in a sample matrix is provided. The system includes a sensor assembly according to the embodiments disclosed herein for sensing an analyte in a sample matrix, a signal processing unit configured to process sensor signals received from the electrode assembly, and a property determination unit configured to, based at least in part on the sensor signals processed from the electrode assembly, determine the property of the analyte in the sample matrix.

In certain embodiments, a method for determining a property of an analyte in a sample matrix is provided. The method includes: providing a sensor assembly including an electrode assembly configured or configurable to define one or more active test electrodes including one or more active electrodes of the set of one or more test electrodes, which one or more active test electrodes contributes to the sensor signal; adjusting a quantity of the analyte provided to the one or more active test electrodes, per unit time, for said interaction based at least in part on an analyte characteristic; processing signals received from an electrode assembly including a set of one or more test electrodes configured to interact with the analyte; and determining the property of the analyte in the sample matrix, based at least in part on the sensor signals processed from the electrode assembly, determine the property of the analyte in the sample matrix.

Certain embodiments of the present disclosure provide a sensor assembly for sensing an analyte which can adjust the amount of the analyte provided to the one or more active test electrodes. By adjusting a quantity of the analyte provided to the active test electrode(s), per unit time, for said interaction based at least in part on an analyte characteristic, embodiments provide sensor assemblies which have improved sensor functionality, including a dynamic sensing range, and the associated sensitivity and accuracy improvements resulting from dynamic sensing range, and improved operating customizability, such as the slowing down or speeding up of testing without falling below the requirements for performance (such as sensitivity or accuracy).

Accordingly, embodiments provide a sensor assembly in which there can be optimization of the sensing conditions. The sensor assembly can alter detection properties such as the sensitivity or detection limits depending at least in part on an analyte characteristic. For example, the adjustment of the quantity of the analyte provided to the active electrode(s), per unit time, for said interaction may be used to move the amount of analyte (concentration) to a range at which the detection is optimal. This could be an amount of analyte where the response of the active electrodes is within a substantially linear or well-defined range than would have otherwise been the case to thereby increase the accuracy of the measurement.

Adjustment of the amount of analyte provided in this way can, in certain embodiments, also be used to increase the sensitivity of the sensor, as required. For example, should an analyte characteristic indicate a low degree of activity (e.g., a low concentration), then the sensor assembly can be configured to increase sensitivity by adjusting a quantity of the analyte provided to the active electrode(s), per unit time, for said interaction based at least in part on the analyte characteristic so that the low concentration can be detected and/or the accuracy can be increased. For example, where the analyte characteristic indicates that the sensor assembly may be close to a lower detection limit, the amount of analyte per unit time provided to the set of test electrode(s) may be increased thereby increasing the signal. This can, for example, be achieved by increasing the number/surface area of test electrodes used to provide the signal, increasing the amount of analyte provided to the analyte surface using the applicator assembly, or a combination of these. In certain embodiments, this adjustment can therefore improve sensitivity and accuracy. Accordingly, in certain embodiments, at least one of the electrode assembly and the applicator assembly is or are configurable to adjust a quantity of the analyte that is delivered to the active electrode arrangement, per unit time, for said interaction based on an analyte characteristic so as to adjust the sensitivity of the electrode assembly.

Certain embodiments thus provide significant advantages over systems and sensor assemblies that cannot adjust the amount of analyte provided to the active electrode(s) per unit time.

Moreover, in embodiments, the provision of this optimization functionality (e.g., the dynamic range that can improve the functionality of the sensors) at device level (e.g., by modifying the test electrode configuration and/or application of sample to the test electrodes) can be particularly advantageous as compared to post-processing of a signal. Without wishing to be bound by theory, adjustment of the electrode assembly and/or the applicator assembly can therefore reduce the reliance on associated electronics to provide improved accuracy and sensitivity (e.g., a dynamic sensing range).

Certain embodiments may also allow for faster analysis or for reduced device usage. For example, for high-concentration analytes, the sample can be delivered faster to the surface since a higher sensitivity is not required. This can increase throughput of samples.

In certain embodiments, the electrode assembly is configured or configurable to vary the quantity of the analyte in contact with the active electrodes, per unit time, for said interaction based at least in part on an analyte characteristic. This can be achieved by, in certain embodiments, the electrode assembly being configured to or configurable to increase or decrease the sensing area provided by the active electrode(s) to thereby increase or decrease, respectively, the amount of analyte provided to the active electrode(s) (per unit time). In other words, the effective sensor surface (the area that is able to interact with the analyte and that is addressed or addressable) provided by the active electrode(s) (effective active test electrode area or surface) can be adjusted to therefore adjust the amount of analyte provided to the active electrode(s) (per unit time). In certain embodiments, this can be achieved by increasing or decreasing the number of electrodes forming the active electrode(s) and/or increasing or decreasing the surface area of the electrodes forming the active electrode(s). This may therefore be an adjustment of the ratio of active to inactive test electrodes (and corresponding control electrodes) and/or a ratio of the effective sensor area to non-addressable sensor area.

As noted, the electrode assembly is configured or configurable to define one of more active test electrodes of the set of one or more test electrodes, which one or more active test electrodes contribute to the sensor signal provided by the set of test electrode(s). In certain embodiments, the electrode assembly is configured or configurable to adjust the number of number of active test electrodes of the set of one or more test electrodes. For example, each electrode of the set of one or more electrodes may be switchable between an active state and an inactive state, the active electrodes being configured to provide the sensor signal based on the interaction with the analyte. The inactive electrodes, if present, do not contribute to the sensor signal. For example, adjustment may include switching at least one inactive electrode to become an active electrode or it may include switching at least one active electrode to become an inactive electrode.

Thus, in certain embodiments, the electrode assembly is configured or configurable to so as to switch at least one active electrode to an inactive electrode, which inactive electrode does not contribute to the sensor signal; and/or where the electrode assembly includes one or more inactive electrodes of the set of electrodes, which one or more inactive electrodes do not contribute to the sensor signal, the sensor assembly is configured or configurable to so as to switch at least one inactive electrode to an active electrode. Thus, only the active electrodes contribute to the sensor signal provided by the set of test electrode(s). In some embodiments, each electrode of the set of test electrode(s) is individually addressable and the sensor assembly switches from an active to an inactive test electrode by no longer addressing that electrode. In an embodiment, this may achieved by disconnecting the active electrode from the signal pathway (e.g., via a switch). The reverse would be the case for switching from an active test electrode to an inactive test electrode. The same is also the case for any control electrodes.

In certain embodiments, the active electrodes may be defined by which electrodes are addressed (e.g., by the sensor assembly, or a system including the sensor assembly). Thus, the electrode assembly is configurable to define the active electrodes by addressing certain electrodes of the set of one or more test electrodes. In certain embodiments, there may be a plurality of test electrodes and at least one of the test electrodes may be addressed to define at least one active electrode. In certain embodiments, at least one electrode may not be addressed so as to define an inactive electrode.

In certain embodiments, the selection of which electrodes are active and, if present, inactive may provide further advantages. In certain embodiments, the sensor assembly and/or electrode assembly may be configured or configurable such that the one or more of the test electrodes that is switched from active to inactive or from inactive to active is based on the configuration or arrangement of the electrodes such that optimal electrode positioning relative to sample matrix delivery is achieved. For example, this may be based on the configuration of the electrodes based on the position relative to the applicator assembly, or more specifically a flow path. Such configurations can provide improved accuracy by selecting the configuration which best accounts for fluid dynamics or flow properties.

For example, taking the case of a Hagen-Poiseuille (pressure-driven, laminar) fully-developed, no-slip-boundary flow, with a parabolic cross-section velocity profile, it may be advantageous to have the active electrodes aligned with the central axis defined by the parabolic cross-section. For example, the active test electrode(s) may be aligned (e.g., parallel to such that the flow passes directly across the electrodes or perpendicular to such that the flow directly impacts the electrodes) with a central axis of a flow path in which such a fluid flow is achieved and those furthest from the central axis may be the first to be switched to inactive electrode(s) first and those nearest the central axis may preferentially be retained as active electrode(s), or the reverse. This can allow for the measurement a particular part of the flow (e.g., the part with the fastest velocity, or the slowest velocity, respectively). It will be appreciated that these advantages may also be achieved where the fluid has a similar, but not perfectly defined, fluid profile. This can be particularly advantageous where the flow is within a flow path with a particular cross-section, such as a circular (including semi-circular) or elliptical shape. In some embodiments, the flow directly impacts the electrodes. For example, the electrode assembly (including at least the set of one or more test electrodes) is angled relative to the central axis (i.e. the one or more test electrodes cross the central axis). Alternatively, it may be that the fluid flow flows over the electrodes such that the one or more test electrodes are parallel to or coaxial with the central axis.

For example, in certain embodiments, the set of one or more test electrodes is spatially distributed about a central axis of symmetry, the applicator assembly being configured such that a path of the sample matrix towards the set of one or more electrodes is parallel or coaxial with the central axis of symmetry. The electrode assembly may be configurable or configured so that switching of at least one active electrode to an inactive electrode includes switching the active electrode or electrodes located furthest from the central axis to an inactive electrode and/or so that switching of at least one inactive electrode to an active electrode includes switching the at least one inactive electrode located closest to the central axis to an active electrode.

In certain embodiments, the set of test electrodes may include a main active electrode which remains as an active electrode and auxiliary or additional test electrodes may be switchable between active and inactive states. A corresponding arrangement may be provided for the control electrodes. This allows the main electrode to be placed in a preferential position for sensing (e.g., in the center of a fluid flow).

In at least some embodiments, the electrode assembly further includes a set of one or more control electrodes, each control electrode being configured for providing a control measurement which is independent of the analyte. In such embodiments, one control electrode can be provided for one of the test electrodes. None of the control electrodes include an analyte interaction portion configured to selectively interact with the analyte. In this manner, each of the control electrodes may permit a control measurement to be taken which is independent of analyte, and in particular independent of the concentration of the analyte. The set of control electrodes may be arranged in any suitable manner, such as in the form of an array. In examples in which the test electrodes are also arranged in an array, the test electrodes array and the control electrodes array may, for instance, extend parallel with each other.

Although the control electrodes do not contribute to the sensor signal, these can provide a control signal which is independent of the analyte in the sample matrix. The electrode assembly may be configured or configurable to define one or more active control electrodes of the set of one or more control electrodes, which one or more active control electrodes contribute to the control signal. The active control electrode(s) may correspond to the test control electrode(s). For example, the electrode assembly may include corresponding active control and test electrode(s) which are activated or left inactive as a pair of electrodes. Thus, as set out above for the set of one or more test electrodes, in embodiments the set of one or more control electrodes may be correspondingly configured or configurable and switchable between these states in a corresponding manner.

In certain embodiments, the electrode assembly is configured and arranged such that the amount of analyte provided to each of the test electrode(s) and the set of one or more control electrodes is substantially equal or equal. In other words, the sensor assembly is arranged with the electrode assembly provided in a flow path (of the applicator assembly) or relative to the applicator assembly and with the electrodes (test and control) arranged in the electrode assembly so that equal or substantially equal amounts of the analyte are provided to the test and control electrodes. This can be based on the flow properties. The equal amounts may be specifically in relation to the active electrode(s) and the corresponding active control electrode(s). In some embodiments, the equal amounts or substantially equal amounts may require that the amount of analyte provided to each of the set or one or more test electrodes is 40 to 60% (of the total analyte provided to the set of one or more test electrodes and the set of one or more control electrodes combined) and that the amount of analyte provided to each of the set or one or more control electrodes is 40 to 60% (of the total analyte provided to the set of one or more test electrodes and the set of one or more control electrodes combined). In some embodiments, the amount supplied to the each of the set or one or more test electrodes or one or more control electrodes may be 45 to 55% of the total provided to the combination, for example, 48% to 52%, for example 49.5% to 50.5%, or for example 50% each.

In certain embodiments, a flow path of the sample matrix towards the electrode assembly defines a central axis and the electrode assembly includes a substrate with the set of one or more test electrodes and the set of one or more control electrodes provided on a first face of the substrate, and wherein the electrode assembly is arranged within the flow path such that first face of the substrate crosses the central axis. Thus, the central axis of the flow path dissects the first face of the substrate. The substrate is thus provided at an angle relative to the central axis (i.e. not parallel to). In one embodiment, the substrate may be perpendicular to the central axis.

This can be a particularly advantageous way of providing the test electrodes, as the center of the fluid flow can impinge on the electrode surface. This improves the likelihood of detection of the analyte, and can correspond to the most uniform part of the flow (as compared to the edges) thereby increasing the accuracy of the measurement.

In one embodiment of this configuration, the set of one or more test electrodes and the set of one or more active electrodes may be provided on a substrate and arranged about a central substrate axis extending perpendicular to the face of the substrate. The set of one or more test electrodes and the set of one or more control electrodes may each define circular or elliptical portions extending around the central substrate axis. These may be concentric. For example, where a plurality of test electrodes and a plurality of control electrodes are provided these may be arranged in the form of concentric circular or elliptical portions and may alternate between test and control electrodes. Each pair of test and control electrodes in such a configuration may be arranged so as to provide equal electrode surface areas. This can improve the accuracy by configuring the control and test electrodes so that they receive the same or nearly the same flow conditions, and correspondingly the same amount of sample matrix.

In one embodiment, the central substrate axis and the central axis of the flow path may be coaxial. This can be particularly advantageous.

In certain embodiments, the electrode assembly is configured or configurable so as to adjust the active electrode surface area thereby adjusting a quantity of the analyte that is delivered to the one or more active test electrodes, per unit time, for said interaction. In some embodiments, this can be achieved as noted above by increasing or decreasing the number of electrodes that are active electrodes.

In certain embodiments, another way of adjusting the amount or quantity of the analyte provided to the one or more active test electrodes, per unit time, is using the applicator assembly. The applicator assembly is configured to enable application of the sample matrix to the set of one or more test electrodes and may be configured to provide the sample matrix to the set of one or more test electrodes. In certain embodiments, the applicator assembly is configured or configurable to adjust the provision of the sample matrix to the set of one or more test electrodes thereby adjusting a quantity of the analyte that is delivered to the one or more active test electrodes, per unit time, for said interaction.

The applicator assembly therefore can be any means or component configured to provide the sample matrix to the electrodes. The applicator assembly may include a fluid pathway (or fluid flow path), such as a channel or conduit, and may be a microfluidic channel arranged to deliver the sample matrix to the electrodes. The applicator assembly may additionally or alternatively include a fluid distribution unit or element, such as a pump, configured to deliver (i.e. actively convey) fluid to the set of one or more test electrodes (and set of one or more control electrodes, where present). Thus the fluid distribution unit may deliver fluid via the fluid pathway or the fluid distribution unit may include a fluid pathway (e.g., if the pathway is capable of effecting movement of the fluid). Where the applicator assembly includes a fluid pathway, the fluid pathway may, in some embodiments, include a circular (including semi-circular) cross-section, such as a cylinder, or elliptical cross-section.

The applicator assembly can thus be used as a means for adjusting the amount or quantity of the sample matrix (and thus the analyte) to the electrode assembly. This provides a relatively straightforward means by which the dynamic range discussed above can be achieved and can provide fine control over the amount of analyte provided to or seen by the electrodes. For example, where there is a low concentration of analyte, the applicator assembly may provide or enable the provision of less sample matrix (and analyte) to the electrode assembly such that more time is provided for diffusion to occur. This can increase sensitivity.

Certain embodiments can also therefore be used to speed up measurement time, as required. For example, where the concentration is high or at the higher end of the measurement spectrum (i.e. relative to the sensor assembly's measurement range), sensitivity is less important such that the amount of sample per unit time can be reduced. In this way, fluid flow across the electrode assembly can be sped up to reduce measurement time or the amount of total sample can be reduced to preserve sample or reduce total measurement time.

In certain embodiments, the applicator assembly is a fluidic assembly configured to deliver the sample matrix (containing the analyte) to the electrode assembly. This may be via a flow path. In certain embodiments, the applicator assembly is configured or configurable to adjust the flow rate of the sample matrix and analyte over the set of one or more test electrodes thereby adjusting a quantity of the analyte that is delivered to the one or more active test electrodes, per unit time, for said interaction.

In certain embodiments, the means of adjusting the amount of analyte provided to the electrode assembly set out above can be combined. For example, certain embodiments may provide an arrangement in which the applicator assembly is configured or configurable to adjust the provision of the sample matrix (and analyte) to the set of one or more test electrodes and in which the electrode assembly is configured or configurable to adjust the number of number of active test electrodes or the surface area of the active electrodes of the set of one or more test electrodes. This can provide a broad dynamic range and customisability. For example, one of the electrode and applicator assembly can be used to provide coarse adjustments and the other can be used to provide fine adjustments.

The adjustment is based on an analyte characteristic. In certain embodiments, this can be selected from the concentration of the analyte in the sample matrix, the diffusion constant of the analyte (e.g., rate of diffusion measured in m²/s) in the sample matrix, or a combination thereof. The analyte characteristic on which the adjustment is based can be measured or estimated.

For example, the analyte characteristic can be measured or estimated by the sensor assembly as part of an initial measurement process. In this way, the sensor assembly may be optimized based on an initially detected or estimated analyte characteristic. In other words, at least one of the electrode assembly and the applicator assembly is or are configurable to adjust a quantity of the analyte provided to the active electrode(s), per unit time, for said interaction based at least in part on an analyte characteristic determined by the sensor assembly. This provides a responsive system that can provide the abovementioned benefits for individual samples.

In certain embodiments, the sensor assembly is configured to obtain an initial indication of the analyte characteristic and provide an initial signal corresponding to the initial indication of the analyte characteristic, such that the initial indication can be determined. The sensor assembly is then configured or configurable to adjust a quantity of the analyte provided to the active electrode(s), per unit time, for said interaction based at least in part on the initial signal. In one embodiment, wherein the analyte characteristic is concentration of the analyte and wherein the sensor assembly is configured to provide an initial signal corresponding to the concentration of the analyte. In some embodiments, the initial indication may be provided by the set of test electrodes. In other embodiments, this may be provided by a separate sensor (e.g., a further set of test electrodes).

By initial indication, it is meant that an initial measurement (determined or estimated) of the characteristic (e.g., concentration). In some embodiments, the initial indication may be less accurate or rely on less information that a normal measurement. For example, it may be that the measurement time for the initial determination is quicker than a standard measurement time for a sample. Alternatively, it may be that the initial measurement is identical to a normal measurement and that it only differs in that the normal measurement is carried out under optimal sensor configurations. That is, the initial measurement may use a default device configuration (e.g., electrode assembly and/or applicator assembly configuration), whereas a normal measurement is carried out once the device has been configured based on the analyte characteristic (e.g., after the initial measurement has been used to adjust the amount of analyte provided to the test electrodes).

For example, this may enable a sample having an unknown concentration to be provided to the sensor assembly. The sensor assembly may then make an initial determination or estimate of the concentration. The sensor assembly can then be reconfigured based on this initial determination or estimate of the concentration to ensure that the amount of analyte provided to the sensor assembly per unit time brings the expected concentration provided to the electrodes to an optimal value (or range) such that it is within a particular (or optimal) operating range of the sensor assembly. For example, this might be an optimized concentration range within which the accuracy of the measurement is higher or may be used to increase the sensitivity of the measurements. This flexibility can allow the sensor assembly to be used with varying concentration ranges without compromising the accuracy of the measurements, for example.

Although in some embodiments the sensor assembly may initially determine or estimate the characteristic and base the adjustment on this characteristic, this need not be necessary and the analyte characteristic may be determined or estimated externally to the sensor assembly.

The terms “analyte concentration” or “concentration of the analyte” as used herein may, in certain embodiments, refer to the activity of the analyte. The activity of the analyte may provide a measure of the effective concentration of the analyte in a sample matrix. The activity may assist to account, for instance, for analyte-analyte interactions in the sample matrix, which may become more relevant at higher analyte concentrations. The above-identified “concentration” terms are nonetheless used herein for convenience.

The analyte may, for example, be selected from a molecular species, a metal ion, a virus, and a microorganism. Particular mention is made of biomarkers, such as a cytokine or a hormone, since these have relevance in the context of patient monitoring, and diagnostic testing. The analyte may, for instance, be a hormone selected from an eicosanoid, a steroid, an amino acid, amine, peptide or protein.

In a non-limiting example, the test electrodes may include analyte interaction portions defined by capture species provided adjacent a surface of the respective test electrode. In such an example, the capture species are configured to selectively interact with the analyte. Any suitable capture species can be selected for this purpose, according to the analyte which is intended to be sensed by the sensor assembly. For example, the capture species may include an antibody with specificity for a particular antigen. In such an example, the analyte may take the form of the antigen. More generally, the capture species may, in some embodiments, include at least one selected from a protein, a peptide, a carbohydrate, and a nucleic acid. The protein may, for example, be an enzyme, such as an enzyme having specificity for the analyte. In other non-limiting examples, the protein is an antibody. In the latter case, the analyte may be an antigen which is selectively bound by the antibody. The capture species may, for instance, include or be defined by an antigen. In this case, the analyte may be a species, such as an antibody, which is selectively bound by the antigenic capture species. The antigen may be or include, for example, a protein, a peptide, a carbohydrate, such as a polysaccharide or glycan.

In an embodiment, the capture species includes an aptamer. An aptamer may be defined as an oligonucleotide or peptide configured to bind the analyte. Such an aptamer may, for example, be configured to interact with, for example bind, various analyte types, such as small molecules, for example amino acids or amines, proteins, metal ions, and microorganisms. In some non-limiting examples, the aptamer is functionalized with an electro-active moiety, for example a redox-active moiety, and is configured such that a conformational change of the aptamer upon selectively interacting with, for example binding, the analyte causes a change in the proximity of the electro-active moiety with respect to the surface of the respective test electrode. Particularly in examples in which the test electrodes are configured for determining a change in current associated with the selective interaction with the analyte, such a change in proximity of the electro-active moiety with respect to the surface of the respective test electrode can cause, or at least contribute to, the determined current change. Thus, the aptamer being functionalized with such an electro-active moiety can assist with amperometric sensing of the analyte. The proximity change resulting from the aptamer interacting with, for example binding, the analyte could, for instance, result in the electro-active moiety moving closer to the surface of the respective test electrode than when the aptamer is not interacting with the analyte. In such examples, electron transfer between the electro-active moiety and the respective test electrode may become faster, such as to contribute to an increase in current in the respective test electrode upon interaction between the analyte and the aptamer. In alternative non-limiting examples, the proximity change resulting from the aptamer interacting with, for example binding, the analyte could result in the electro-active moiety moving further from the surface of the respective test electrode than when the aptamer is not interacting with the analyte. In such examples, the aptamer may be regarded as being conformationally configured in the absence of the analyte such that the electro-active moiety, for example redox-active moiety, is proximal to, or even in contact with, the test electrode surface, thereby providing a baseline signal. In such cases, a decrease in current in the respective test electrode upon interaction between the analyte and the aptamer may be observed. Thus, the greater the concentration of analyte, the greater the decrease in the current. Any suitable electro-active moiety may be included in the aptamer for this purpose, such as methylene blue.

In some embodiments, each test electrode surface is functionalized with the capture species. Such functionalization can be achieved in any suitable manner, such as by covalently or non-covalently immobilizing the capture species to the surface. For example, thiol-terminated capture species, such as a thiol-terminated aptamer, can be immobilized, for example grafted, onto the surface of a noble metal, for example gold, electrode.

More generally, the test electrode arrangement and the set of control electrodes are arranged to receive a sample matrix. Sample matrix refers to the sample as a whole, including the analyte if present. Thus, it may include a carrier (such as a liquid) and the analyte. The sample matrix may be, for example, blood, urine, sweat, tears, etc., and may (potentially) contain the analyte. In an embodiment, the sample matrix is a liquid.

A system for determining a property of an analyte in a sample matrix includes any of the sensor assembly embodiments disclosed herein, together with a signal processing unit configured to process sensor signals received from the electrode assembly and a property determination unit configured to, based at least in part on the sensor signals processed from the electrode assembly, determine the property of the analyte in the sample matrix. In some embodiments, the system is for determining the concentration of an analyte in a sample matrix. The system can be configured to determine the concentration of the analyte in the sample matrix based at least in part on the sensor signal.

As noted above, the sensor assembly can, in some embodiments, be configured to provide an initial signal corresponding to the analyte characteristic. Thus, the system can be configured to obtain an initial indication of the analyte characteristic based on the initial signal. In certain embodiments, the system may therefore calculate or estimate the initial indication of the analyte characteristic. This may provide a rough or initial guide to the expected value of the analyte characteristic. Based on this initial indication, the system can be configured or configurable to adjust a quantity of the analyte provided to the one or more active test electrodes, per unit time, for said interaction based at least in part on the initial indication of the analyte characteristic. In some embodiments, the system itself may be configured to determine the initial indication of the analyte characteristic. This may be carried out by the property determination unit and the signal proceeding unit or may be a separate processing means.

The property determination unit may be a concentration determination unit in certain embodiments. The property determination unit may, in certain embodiments, be configured to determine the property based on (at least) the absolute change in signals in the test electrode (e.g., a pair that includes a test electrode and a control electrode), and/or the rate of change of the signals.

In certain embodiments, the system further includes a processing unit. The processing unit may incorporate the property determination unit and/or the signal processing unit or may be in addition to one or both of these. The processing unit may be configured to adjust the amount of analyte provided to one or more active test electrodes by controlling the operation of the sensor assembly. For example, the processor unit may control operation of the applicator assembly and/or electrode assembly. For example, the processor unit may determine which electrodes are addressed (test and control, where present).

The signal processing unit, the property determination unit and processor may be implemented in any suitable manner, with software and/or hardware, to perform the various functions required. One or all of the units may, for example, employ one or more microprocessors programmed using software (for example, microcode) to perform the required functions. Examples of processor components that may be employed in various embodiments of the present disclosure include, but are not limited to, conventional microprocessors, application specific integrated circuits (ASICs), and field-programmable gate arrays (FPGAs).

In various implementations, the signal processing unit, property determination unit and/or processor may be associated with one or more non-transitory storage media such as volatile and non-volatile computer memory such as random-access memory (RAM), programmable read-only memory (PROM), erasable PROM (EPROM), and electrically EPROM (EEPROM). The non-transitory storage media may be encoded with one or more programs that, when executed on one or more processors and/or controllers, perform the required functions. Various storage media may be fixed within a processor or controller or may be transportable, such that the one or more programs stored thereon can be loaded into the signal processing unit, property determination unit and/or processor.

In some non-limiting examples, the system includes a user interface, such as a display, for communicating the analyte property determined by the property determination unit. Alternatively or additionally, the system may include a communications interface device, such as a wireless transmitter, configured to transmit the analyte concentration determined by the property determination unit to an external device, such as a personal computer, tablet, smartphone, remote server, etc.

Methods for determining a property of an analyte in a sample matrix may therefore include the steps of:
a. providing a sensor assembly including an electrode assembly configured or configurable to define one or more active test electrodes including one or more active electrodes of the set of one or more test electrodes, which one or more active test electrodes contributes to the sensor signal;
b. adjusting a quantity of the analyte provided to the one or more active test electrodes, per unit time, for said interaction based at least in part on an analyte characteristic;
c. processing signals received from an electrode assembly including a set of one or more test electrodes configured to interact with the analyte; and
d. determining the property of the analyte in the sample matrix, based at least in part on the sensor signals processed from the electrode assembly, determine the property of the analyte in the sample matrix.

In certain embodiments, the property of the analyte is the concentration of the analyte in a sample matrix.

In certain embodiments, the method further includes determining an initial indication of the analyte characteristic; and the step of adjusting a quantity of the analyte provided to the one or more active test electrodes, per unit time, for said interaction is based at least in part on the initial indication of the analyte characteristic. This may be achieved by receiving an initial sensor signal from the sensor assembly and determining the initial indication of the analyte characterise based on the initial sensor signal. As noted above, in some embodiments, the analyte characteristic is the concentration of the analyte in the sample matrix.

In a further aspect, a sensor assembly for sensing an analyte in a sample matrix includes an electrode assembly and a flow path. The electrode assembly includes a set of one or more test electrodes, the set of one or more test electrodes including an analyte interaction portion configured to interact with the analyte and provide a sensor signal based on said interaction, and a set of one or more control electrodes, the set of one or more control electrodes providing a control electrode area configured for providing a control measurement which is independent of the analyte. The flow path is configured to provide the sample matrix to the electrode assembly. The electrode assembly is configured and arranged in the flow path such that the amount of sample matrix provided to each of the set or one or more test electrodes and the set of one or more control electrodes is substantially equal or equal. A control electrode area may be provided for each of the test electrodes.

Embodiments of this further aspect can advantageously provide improved accuracy of a measurement. The configuration of the electrodes (e.g., the shape, ratio of the surface area between the control and test electrode(s)) and the configuration relative to the flow path ensures that the control and test electrode(s) receive the same amount of sample matrix (i.e. the same amount of analyte). This ensures that the control electrode is subject to the same conditions as the test electrodes, which should ensure that any other effects (e.g., degradation or interference) on the test electrode is also occurring on the control electrode. This can increase the accuracy of the differential between the measurement of the test electrode and the corresponding control electrode.

In certain embodiments, the electrode assembly is configured and arranged in the flow path such that the amount of analyte provided to each of the set or one or more test electrodes is 40 to 60% of the total analyte provided to the set of one or more test electrodes and the set of one or more control electrodes combined and the electrode assembly is configured and arranged in the flow path such that the amount of analyte provided to each of the set or one or more control electrodes is 40 to 60% of the total analyte provided to the set of one or more test electrodes and the set of one or more control electrodes combined. As noted above, in some embodiments, the amount supplied to the each of the set or one or more test electrodes or one or more control electrodes may be 45 to 55% of the total provided to the combination, for example, 48% to 52%, for example 49.5% to 50.5%.

In certain embodiments, the flow path of the sample matrix towards the electrode assembly defines a central axis, wherein the electrode assembly includes a substrate with the set of one or more test electrodes and set of one or more control electrodes provided on a first face of the substrate, and wherein the electrode assembly is arranged within the flow path such that the first face of the substrate crosses the central axis of the flow path. In other words, the central axis of the flow path dissects the first face of the substrate. In embodiments, the first face is substantially perpendicular or perpendicular to the central axis of the flow path. This can be particularly advantageous, as detailed above.

In certain embodiments, the set of one or more test electrodes and the set of one or more control electrodes is spatially distributed about a central axis of symmetry, and wherein the flow path of the sample matrix towards the set of one or more test electrodes is coaxial or parallel with the central axis of symmetry. This can center the electrode assembly on the center of the flow of the sample matrix, thereby further improving the accuracy of the measurements.

In one embodiment of this configuration, the set of one or more test electrodes and the set of one or more active electrodes may be provided on a substrate and arranged about a central substrate axis extending perpendicular to the face of the substrate. The set of one or more test electrodes and the set of one or more control electrodes may each define circular or elliptical portions extending around the central substrate axis. These may be concentric. For example, where a plurality of test electrodes and a plurality of control electrodes are provided these may be arranged in the form of concentric circular or elliptical portions and may alternate between test and control electrodes. Each pair of test and control electrodes in such a configuration may be arranged so as to provide equal electrode surface areas. Embodiments provide a particularly advantageous configuration which centers the electrode on the center of the flow path of the sample matrix. As noted above, this can help to ensure that the distribution of analyte within normal fluid flows in flow paths match the electrode structure to provide equal or substantially equal sample matrix to the test and control electrode(s).

In one embodiment, the central substrate axis and the central axis of the flow path may be coaxial. This can be particularly advantageous as the sets of electrode(s) are centered on the flow path.

In certain embodiments, the electrode assembly is configured or configurable so as to adjust the active electrode surface area thereby adjusting a quantity of the analyte that is delivered to the one or more active test electrodes, per unit time, for said interaction. In some embodiments, this can be achieved as noted above by increasing or decreasing the number of electrodes that are active electrodes.

In certain embodiments, the flow path of the sample matrix towards the electrode assembly defines a central axis, wherein the electrode assembly includes a substrate with the set of one or more test electrodes and set of one or more control electrodes provided on a first face of the substrate, and wherein the electrode assembly is arranged within the flow path such that flow path is parallel to the first face of the substrate.

In certain embodiments, the test electrodes of the set of one or more test electrodes and the control electrodes or the set of one or more control electrodes are interdigitated and arranged in the flow path such that the flow of analyte across the set of one or more test electrodes and the set of one or more control electrodes is substantially equal or equal.

A system for determining a property of an analyte in a sample matrix may include the sensor assembly of the further aspects, a signal processing unit configured to process sensor signals received from the electrode assembly; and a property determination unit configured to, based at least in part on the sensor signals processed from the electrode assembly, determine the property of the analyte in the sample matrix. In certain embodiments, the property determination unit is for determining the concentration of an analyte in a sample matrix, and the system is configured to determine the concentration of the analyte in the sample matrix based at least in part on the sensor signal. The property determination unit may accordingly be a concentration determination unit.

A computer program including computer program code which is configured, when said computer program is run on one or more physical computing devices, to cause said one or more physical computing devices to implement the methods disclosed herein.

One or more non-transitory computer readable media having a computer program stored thereon, the computer program including computer program code which is configured, when said computer program is run on one or more physical computing devices, to cause said one or more physical computing devices to implement the method disclosed herein.

The description may use the phrases “in an embodiment” or “in embodiments,” which may each refer to one or more of the same or different embodiments. Unless otherwise specified, the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner. Furthermore, for the purposes of the present disclosure, the phrase “A and/or B” or notation “A/B” means (A), (B), or (A and B), while the phrase “A, B, and/or C” means (A), (B), (C), (A and B), (A and C), (B and C), or (A, B, and C). As used herein, the notation “A/B/C” means (A, B, and/or C). The term “between,” when used with reference to measurement ranges, is inclusive of the ends of the measurement ranges.

Various aspects of the illustrative embodiments are described using terms commonly employed by those skilled in the art to convey the substance of their work to others skilled in the art. For example, the term “connected” means a direct electrical connection between the things that are connected, without any intermediary devices/components, while the term “coupled” means either a direct electrical connection between the things that are connected, or an indirect connection through one or more passive or active intermediary devices/components. In another example, the term “circuit” means one or more passive and/or active components that are arranged to cooperate with one another to provide a desired function. Sometimes, in the present descriptions, the term “circuit” may be omitted (e.g., a current mirror circuit may be referred to simply as a “current mirror,” etc.). If used, the terms “substantially,” “approximately,” “about,” etc., may be used to generally refer to being within +/−10% of a target value, e.g., within +/−5% of a target value or within +/−2% of a target value, based on the context of a particular value as described herein or as known in the art.

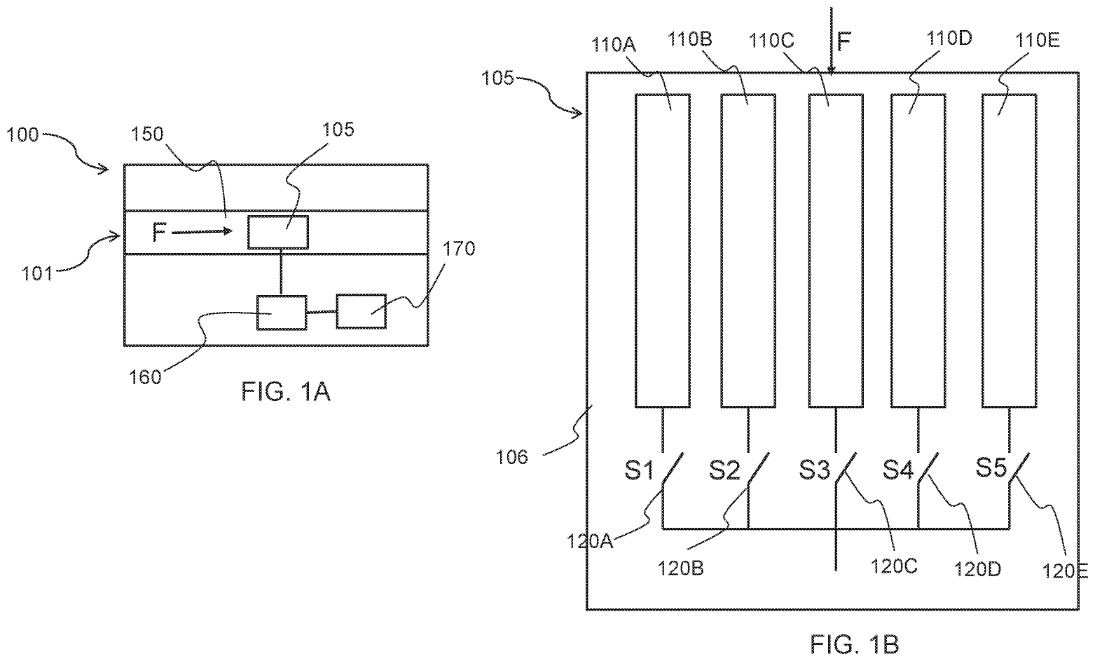

FIGS. 1A and 1B shows an embodiment of a system 100 for determining the concentration of an analyte in a sample matrix according to an example. FIG. 1A shows a schematic view of the system 100, which system 100 includes a sensor assembly 101, a signal processing unit 160 and a processor 170 which acts as a concentration determination unit. The sensor assembly 101 includes an electrode assembly 105 and an applicator assembly including a fluid flow path 150. FIG. 1B shows a schematic plan view of the electrode assembly 105, which includes a set of five test electrodes 110A-110E on a substrate 106 and a set of five corresponding switches 120A-120E. A set of corresponding control electrodes are present but not depicted. In this non-limiting example, each control electrode surface area is the same as the test electrode surface area (specifically the area functionalized with a capture species of each test electrode).

In this embodiment, the system 100 is configured to determine the concentration of an analyte in a liquid sample matrix. As such, the applicator assembly includes a fluid flow path 150 and the electrode assembly 105 is located within the fluid flow path 150. Arrow F indicates the direction of fluid flow within the fluid flow path 150 in both FIG. 1A and FIG. 1B.

In the non-limiting example depicted in FIGS. 1A and 1B, the test electrodes 110A-110E and the control electrodes (not depicted) are provided, for example formed using semiconductor lithographic techniques, on a common substrate 106, for example a silicon substrate 106. The test electrodes 110A-110E are rectangular electrodes and are arranged in parallel (relative to the elongate part of the electrode) in an array such that there is a single middle test electrode 110C, two intermediate test electrodes 110B, 110D on either side of the middle test electrode 110C and two outer test electrodes 110A, 110E on either side of the array. A central axis of symmetry thus runs through the middle test electrode 110C, which in this embodiment is parallel to the flow of fluid through the fluid flow path 150. Thus, the electrode assembly 105 in this embodiment is arranged such that the flow of liquid sample matrix through the fluid flow path 150 is centered on the middle test electrode 110C.

Each electrode 110A-110E is connected to a corresponding individual switch 120A-120E and each test electrode 110A-110E can individually be connected or disconnected from the signal processing unit 160 by the switch so that the test electrode 110A-110E is no longer addressable. Thus, switches 120A-120E determine whether each of the test electrodes is active, and contributes towards the sensor signal sent from the test electrode(s) 110A-110E to the signal processing unit 160, or inactive, and does not contribute to the sensor signal. The switches 120A-120E also determine whether a corresponding control electrode (not shown) is connected to the signal processing unit 160.

The set of test electrodes 110A-110E have areas functionalized with capture species configured to interact with, for example bind, the analyte. In this case, the capture species includes an aptamer.

A sample matrix containing analyte is introduced into the fluid flow path 150. The set of test electrodes 110A-110E, and specifically those which are active (discussed in more detail below), will generate a sensor signal based on an interaction with an analyte. This sensor signal is transmitted to the signal processing unit 160 where it is processed. In this non-limiting embodiment, there is a processor 170 located within the system 100, which processor 170 determines the concentration of the analyte in the sample matrix based on the processed sensor signal. The concentration is then output to an external device via a communications module (not shown).

Two different methods of using the system 100 of FIGS. 1A and 1B will now be described.

The first method involves active feedback whereby the system 100 is used to obtain an initial measurement of the concentration, and processor 170 determines the optimal configuration of the sensor assembly 101 for the concentration detected in the initial measurement. In more detail, a fluid sample (i.e. the sample matrix) is provided to the applicator assembly by providing a continuous flow of the fluid sample to fluid flow path 150. This is delivered to the electrode assembly 105. In a non-limiting example, electrode assembly 105 is in a default initial configuration where three of the five test electrodes are active. In particular, the middle test electrode 110C and the adjacent intermediate test electrodes 1106 and 110D are all active due to their corresponding switches 120B, 120C, 120D being closed. The switches 120A, 120E for the outermost test electrodes 110A, 110E remain open such that the outermost test electrodes 110A, 110E are not addressable.

The flow of fluid sample matrix across the active test electrodes 110B, 110C, 110D generates an initial sensor signal due to the specific binding of the analyte to the aptamer. This initial sensor signal is received by the signal processing unit 160, processed, and then transmitted to the processor 170. Processor 170 determines an initial indication of the concentration of the aptamer in the fluid sample matrix. Processor 170 then determines whether and how to adjust the amount of analyte per unit time provided to active electrodes. In this embodiment, this adjustment of the amount of analyte per unit time is achieved by designating or de-designating each of the test electrodes 110A to 1106 as active electrodes.

In one example, if the processor 170 determines that there is a low concentration of the analyte in the sample matrix, to increase the sensitivity of the sensor assembly 101 the processor 170 can increase the number of active electrodes. In this particular embodiment, this can be achieved by switching the outermost test electrodes 110A, 110E to be active by closing their corresponding switches 120A, 120E. In this way, the total effective sensor area is increased such that the total sensor signal output by the electrode assembly 105 is increased. In other words, by increasing the number of the active test electrodes, the amount of analyte per unit time provided to active test electrodes (as a whole) is increased. This increases the sensitivity of the sensor assembly 110.

If instead the processor 170 determines that there is a high concentration of the analyte in the sample matrix, the reverse may occur. In particular, the processor 170 may determine that only the middle test electrode 110C need be addressed and so the adjacent, intermediate test electrodes 110B, 110D may be switched from active to inactive by opening their corresponding switches 120B, 120D.

The second method does not require an initial measurement by the sensor assembly or system. Instead, a user may provide a fluid sample to the system 100 for which an estimate of the analyte concentration is known. A user may therefore manually configure the sensor assembly 110 or enter the estimate into the system 100 (e.g., via a user interface) such that the processor 170 can configure the sensor assembly 110 accordingly. The adjustment is then carried out in the manner described for the first method.

In the above examples of the method of use of the system 100 of FIGS. 1A and 1B, the adjustment of the amount of fluid provided to the active electrodes of the electrode assembly 105 is determined by the number of active test electrodes (and thus the total effective sensor area). However, in a further example, the system 100 may further include a pump (not shown) which determines the fluid flow of the sample matrix through the fluid flow path 150. The pump may, in certain embodiments, be controlled by processor 170. Based on the analyte concentration, the fluid flow may be adjusted in combination with the number of active test electrodes 110A-110E to provide varying degrees of control over the amount of fluid provided to the active electrodes of the electrode assembly 105 per unit time.

As discussed above, in the embodiment of FIGS. 1A and 1B and the methods discussed in conjunction with this embodiment, the test electrodes 110A-110E are provided in an array which has a central axis of symmetry through the middle test electrode 110C and which is parallel to the fluid flow through the fluid flow path 150 (which passes over the electrode assembly 105). This allows the center of the fluid flow to pass over the middle test electrode 110C. This can be advantageous for particular flows as particular fluid dynamics can result in a non-linear fluid velocity when examining a cross-section through the fluid pathway. For example, in the case of a Hagen-Poiseuille (pressure-driven, laminar) fully-developed, no-slip-boundary flow with a parabolic cross-section velocity profile centered on, centring the flow on the middle test electrode 110C allows for the test electrode to be located in the highest part of the parabola. The subsequent preferential addressing of the electrodes closest to the central axis (i.e. middle test electrode 110C most preferentially, followed by the adjacent intermediate electrodes 110B, 110D with the outer test electrodes 110A, 110E being the last to be converted to active electrodes ensures that the electrodes closest to this parabola are addressed. This allows the detection of the analytes in the center of the parabolic profile (with respect to the direction orthogonal to the flow), where velocity can be the highest. The system 100 also allows the reverse to be carried out, whereby only the outer test electrodes 110A, 110E are addressed allowing the interrogation of the part of the flow which may have the lowest velocity. The latter may, for example, allow increased time for interaction between an analyte flowing over the electrodes, then the former.

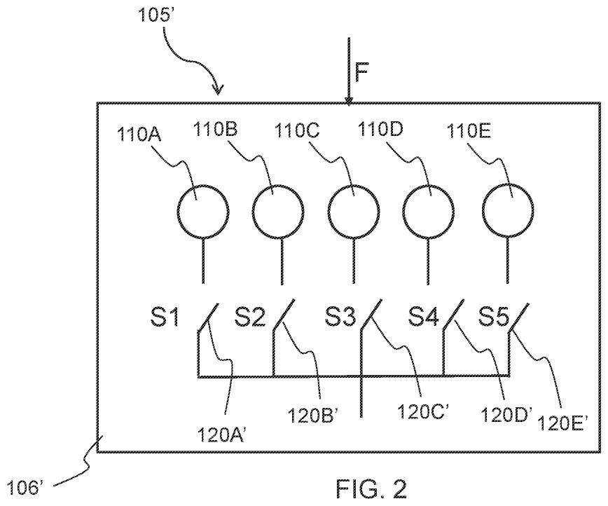

FIG. 2 shows an alternative electrode assembly 105′ for use in system 100. The electrode assembly 105′ has a similar structure to the electrode assembly 105 depicted in FIG. 1A, with the exception of the shape of the test electrodes 110A-110E and the control electrodes (not shown). Accordingly, the electrode assembly 105′ includes a set of five test electrodes 110A′-110E′ on a substrate 106′ and a set of five corresponding switches 120A′-120E′. A set of corresponding control electrodes are present but not depicted.

In this non-limiting example, the test electrodes 110A-110E are circular electrodes and are arranged in a single plane (extending perpendicular relative to the flow direction) in an array such that there is a single middle test electrode 110C′, two intermediate test electrodes 110B′, 110D′ on either side of the middle test electrode 110C′ and two outer electrodes 110A′, 110E′ on either side of the array. A central axis of symmetry thus runs through the middle test electrode 110C′ parallel to the flow of fluid through the fluid flow path 150. Thus, the electrode assembly 105′ in this embodiment is arranged such that the flow of fluid sample matrix is centered on the middle test electrode 110C′. The control electrodes are correspondingly arranged (not shown).

Each test electrode 110A′-110E′ is connected to a corresponding individual switch 120A′-120E′ and each test electrode 110A′-110E′ can individually be connected or disconnected from a signal processing unit by the corresponding switch 120A′-120E′ so that the test electrode 110A-110E is no longer addressable.

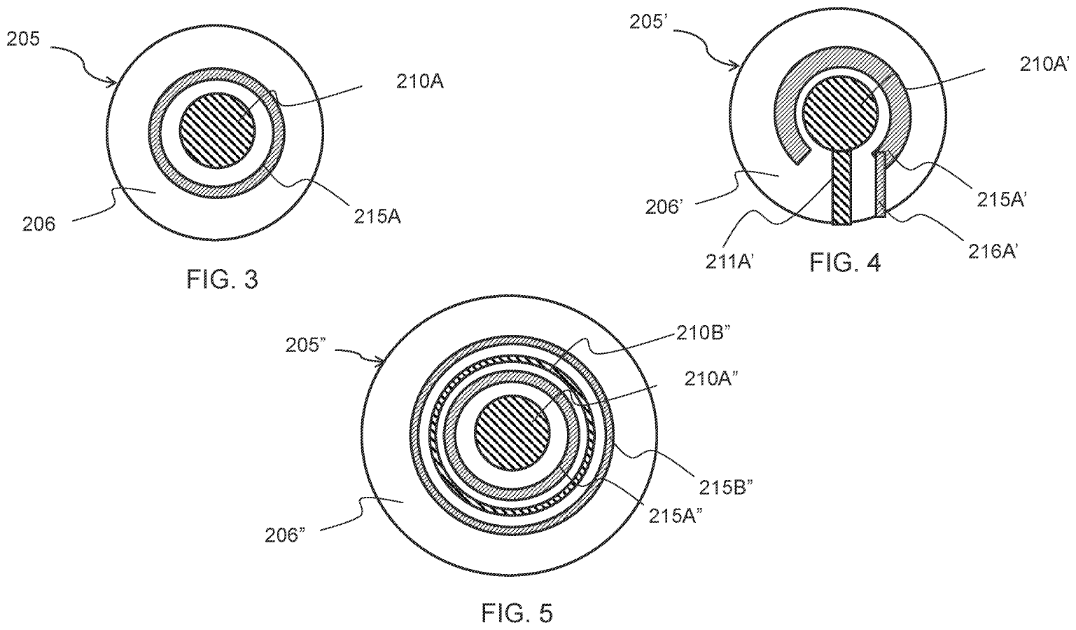

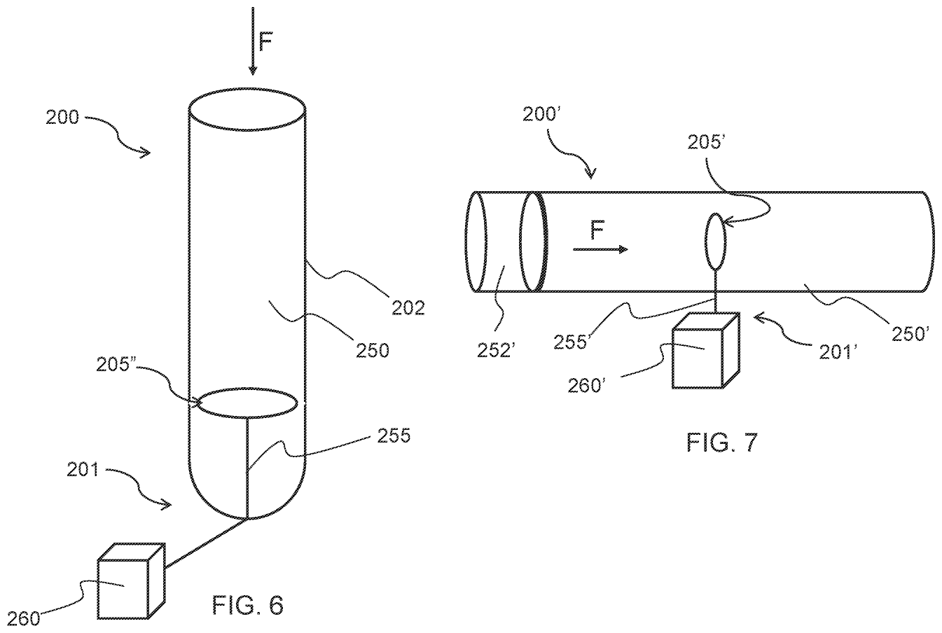

FIGS. 3 to 5 depict schematic top plan views of three further example electrode assemblies 205, 205′, 205″. In a non-limiting embodiment, these electrode assemblies 205, 205′, 205″ can be employed in the system 100 of FIG. 1, in the manner of the electrode assembly 105′ of FIG. 2, for example. In alternative embodiments, embodiment, these electrode assemblies 205, 205′, 205″ can be employed in the systems 200, 200′ of FIGS. 6 and 7, discussed in more detail below.

FIG. 3 depicts an electrode assembly 205 including a substrate 206 on which a test electrode 210A and a control electrode 215A are provided. In this embodiment, the substrate 206 is circular and the test electrode 210A and control electrode 215A are circular and are arranged concentrically. In particular, the test electrode 210A has a circular shape and is provided in the center of the substrate 206 and the control electrode 215A is circular and arranged around (and spaced apart from) the test electrode 210A. The electrodes 210A, 215A are concentric around a central axis extending perpendicular to the face of the substrate 206. The surface area of the control electrode 215A and the surface area of the test electrode 210A are equal. Although not shown, each of the test electrode 210A and control electrode 215A are electrically connected to a signal processing unit (not shown) by an electrical connection through a via in the substrate 206.

It will be appreciated that the inverse arrangement could also be provided; that is, in some embodiments, the control electrode could be provided as the central electrode, with the test electrode being provided around the control electrode.

FIG. 4 depicts a further electrode assembly 205′ with a similar configuration of a substrate 206′ on which a central circular test electrode 210A′ surrounded by a circular portion of a control electrode 215A′ is provided. The effective sensor area of each of the test 210A′ and control 215A′ electrodes is the same. In this embodiment, the electrical connections 211A′, 216A (signal traces) are run in the same layer as the electrodes. To avoid contact with the fluid sample, a passivation layer may be provided on top of the traces.

FIG. 5 depicts a further electrode assembly 205″ with a similar configuration. In this embodiment, there are plural test electrodes 210A″, 210B″ in the set of test electrodes and plural control electrodes 215A″, 215B″ in the set of control electrodes. These are arranged concentrically as in the embodiment of FIGS. 3 and 4. In particular, the first test electrode 210A″ has a circular shape and is provided in the center of a substrate 206″ and the first control electrode 215A″ is circular and arranged around (and spaced apart from) the first test electrode 210A″. The second test electrode 210B″ is also circular and is arranged around the first control electrode 215A″ and the second control electrode 210B″ is also circular and arranged as the outer electrode around the second test electrode 210B″. The electrodes 210A″, 210B″, 215A″, 215B″ are concentric around a central axis extending perpendicular to the face of the substrate 206″. The surface area of the first control electrode 215A″ and the surface area of the first test electrode 210A″ are equal and the surface area of the second control electrode 215B″ and the surface area of the second test electrode 210B″ are equal. The surface area of all of the electrodes 210A″, 210B″, 215A″, 215B″ may be the same, or each pair of electrodes (i.e. a pair of one test electrode 210A″, 210B″ and one control electrode 215A″, 215B″) may be the same but different from the other pairs.

These embodiments are particularly advantageous as they can be used in a circularly symmetric flow path. Molecular transport will determine whether the analytes get a chance to bind to the sensing electrodes and not bind to the control electrodes. This particular configuration may include the likelihood that both electrodes will have as close to “the same flow conditions” as possible. For instance, if non-specific binding occurs, it may occur the same amount on the control and the sensing electrodes, making it easier to subtract the non-specific binding signal on the control electrode from the non-specific binding on the sensor electrode.

As will be explained in more detail below, the electrode assemblies 205, 205′ of FIGS. 3 and 4 can be used in a system in which the amount of analyte provided to the test electrode 210A, 210A′ is adjusted by an applicator assembly. The electrode assembly 205″ of FIG. 5 may be adjusted by the electrode assembly itself (in embodiments, in the manner of the system 100 of FIG. 1A) and/or by an applicator assembly.

FIGS. 6 and 7 depict embodiments of systems 200, 200′ configured to determine the concentration of an analyte in a sample matrix according to an example.

FIG. 6 shows a schematic perspective view of system 200, which system 200 includes a sensor assembly 201, an electronics unit 260 (including a signal processing unit and a processor which acts as a concentration determination unit). Due to the presence of plural electrodes, the sensor assembly 201 includes an electrode assembly 205″ and an applicator assembly includes a fluid flow path 250. In this embodiment, the electrode assembly is the electrode assembly 205″ depicted in FIG. 5. However, it will be appreciated that, in other embodiments, the electrode assemblies 205, 205′ of FIGS. 3 and 4 could be used in place of the electrode assembly 205″.

In this embodiment, the system 200 is provided as a test tube 202 including the sensor assembly 201 and connected to an external electronics unit 260 via an electrical connector 255. The test tube 202 thus defines a fluid flow path 250 which extends along the elongate body of the test tube 202. The electrode assembly 205″ is positioned in the test tube so as to be perpendicular to the fluid flow path 250. In this case, the flow will be flow of the analyte as it diffuses through the fluid in the test tube 202. Arrow F indicates the direction of the flow of the analyte within the fluid flow path 250. This is along the central axis defined by the elongate cylindrical portion of the test tube 202. The face of the substrate 206 is therefore perpendicular to this axis. The fluid flow path 250 defined by the test tube 202 is circularly symmetrical.

In use, adjustment of the amount of analyte in this embodiment can be achieved by activating/addressing one or both of the test electrodes 210A″, 210B″ based on the analyte concentration.

FIG. 7 shows a schematic perspective view of system 200′, which system 200′ includes a sensor assembly 201′, an electronics unit 260′ (including a signal processing unit and a processor which acts as a concentration determination unit). The sensor assembly 201′ includes an electrode assembly 205′ and an applicator assembly includes a fluid flow path 250′ and a pump 252′. In this embodiment, the electrode assembly is the electrode assembly 205′ depicted in FIG. 4. However, it will be appreciated that, in other embodiments, the electrode assemblies 205, 205″ of FIGS. 3 and 5 could be used in place of the electrode assembly 205′.

In this embodiment, the system 200′ provides a continuous flow of fluid sample matrix to the electrode assembly 205′ via pump 252′ along the fluid flow path 250′. In some embodiments, the system 200′ may be provided as an inline sensor assembly 201′ or may be provided in a separate sampling flow path. The electrode assembly 205′ is positioned in the fluid flow path 250′ so as to be perpendicular to the fluid flow F along the fluid flow path 250. The face of the substrate 206′ of the electrode assembly 205′ is perpendicular to the central axis of the fluid flow path 250. The fluid flow path 250, and therefore the flow, is circularly symmetrical and is centered on the center of the face of the substrate 206′.

In use, adjustment of the amount of analyte in this embodiment can be achieved by adjusting the flow rate through the fluid flow path 250′ using the pump 252′ based on the analyte concentration.

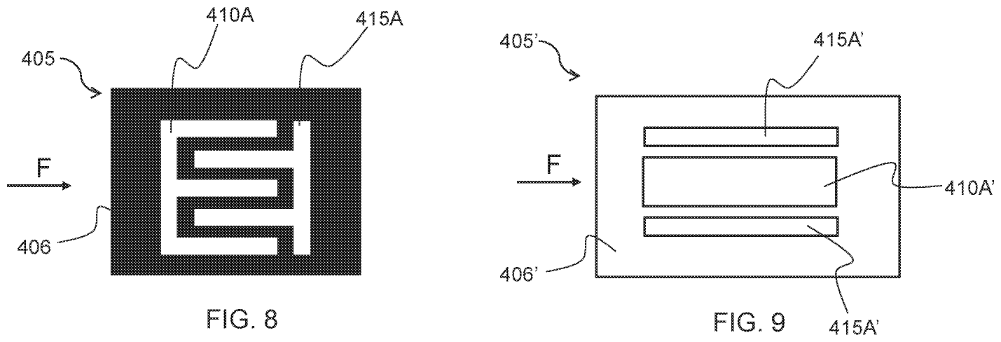

FIGS. 8 and 9 shows electrode assemblies 405, 405′ for positioning in a flow path where the flow direction is across the face of the substrates 406, 406′ such that the flow is parallel to the surface of the substrate 406, 406′ to increase the likelihood that the electrodes on each substrate 406, 406′ will see identical flows of fluid sample matrix (and thus analyte).

The electrode assembly 405 of FIG. 8 includes a substrate 406 on which is provided a test electrode 410A and a control electrode 415A. The two electrodes 410A, 415A are of equal total surface area. The two electrodes 410A, 415A are interdigitated and arranged relative to the flow direction F so that flow conditions, and thus the amount of analyte, are substantially equal or equal.

The electrode assembly 405′ of FIG. 9 includes a substrate 406′ on which is provided a test electrode 410A and a control electrode formed of two control electrode portions 415A′. The test electrode 410A′ has a sensor area that is equal to the total of both of the control electrode portions 415A′. The electrodes 410A, 415A′ are interdigitated and arranged relative to the flow direction F so that flow conditions, and thus the amount of analyte, are substantially equal or equal. The area and shape for 410A′ and 415A′ are chosen so that, during a measurement phase, the same amount of a given target analyte would adhere to test electrodes 410A′ as it would to control electrodes 415A′.

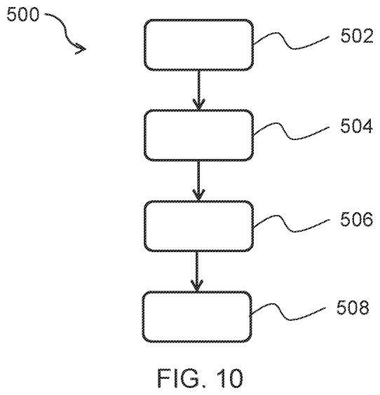

FIG. 10 shows a flowchart of a method 500 according to an example. The method 500 is for determining a concentration of an analyte in a sample matrix. The method 500 includes, in block 502, providing a sensor assembly including an electrode assembly configured or configurable to define one or more active test electrodes including one or more active electrodes of the set of one or more test electrodes, which one or more active test electrodes contributes to the sensor signal.

In block 504, the method 500 includes adjusting a quantity of the analyte provided to the one or more active test electrodes, per unit time, for said interaction based at least in part on an analyte characteristic.

In block 506, the method 500 includes processing signals received from an electrode assembly including a set of one or more test electrodes configured to interact with the analyte.

The method 500 also includes, in block 508, determining the property of the analyte in the sample matrix, based at least in part on the sensor signals processed from the electrode assembly, determine the property of the analyte in the sample matrix.

Although not shown in FIG. 10, in some embodiments the method 500 may further include determining an initial indication of the analyte characteristic and the step of adjusting a quantity of the analyte provided to the one or more active test electrodes, per unit time, for said interaction is based at least in part on the initial indication of the analyte characteristic.

In some embodiments, the analyte characteristic is the concentration of the analyte.

In a non-limiting example, the determining 508 is based on the absolute change in signals in the test electrode-control electrode pairs, and the rate of change of the signals.

The method 500 may, for example, employ the sensor assembly and/or the system according to any of the embodiments and examples described herein. In particular, the steps of processing 506 and determining 508 of method 500 may be implemented using the signal processing unit and the property determination unit of the systems of the present disclosure.

In certain embodiments, a computer program including computer program code is adapted, when the program is run on a computer, to implement the steps of processing 506 and determining 508 of method 500 according to any of the examples and embodiments described herein. Such a computer may, for example, be included in, or define, the signal processing unit and the property determination unit of the systems of the present disclosure. The computer program may be stored on one or more non-transitory computer readable media. The computer program may include instructions, when executed by one or more physical computing devices such as one or more processors, can cause the one or more processors to implement, execute and/or carry out one or more methods described herein.

More generally, examples and embodiments described herein in respect of the sensor assemblies may be applicable to any of the systems, methods 500 and/or computer program. Similarly, examples and embodiments described herein in respect of the system, method 500 and/or computer program may be applicable to the sensor assemblies.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present disclosure can be better understood from the description, appended claims or aspects, and accompanying drawings. It should be understood that the drawings are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

Numerous other changes, substitutions, variations, alterations, and modifications may be ascertained to one skilled in the art and it is intended that the present disclosure encompass all such changes, substitutions, variations, alterations, and modifications as falling within the scope of the appended select examples. Note that all optional features of the apparatus described above may also be implemented with respect to the method or process described herein and specifics in the examples may be used anywhere in one or more embodiments.

1. A sensor assembly for sensing an analyte in a sample matrix, the sensor assembly comprising:
an electrode assembly comprising a set of one or more test electrodes to interact with the analyte and provide a sensor signal based on said interaction; and
an applicator assembly to enable application of the sample matrix to the set of one or more test electrodes,
wherein the electrode assembly is to define one or more active test electrodes of the set of one or more test electrodes, which one or more active test electrodes contribute to the sensor signal, and
wherein at least one of the electrode assembly and the applicator assembly is to adjust a quantity of the analyte provided to the one or more active test electrodes, per unit time, for said interaction based at least in part on an analyte characteristic.

2. The sensor assembly according to claim 1, wherein the analyte characteristic includes at least one of a concentration of the analyte in the sample matrix or a diffusion constant of the analyte in the sample matrix.

3. The sensor assembly according to claim 1, wherein:
the sensor assembly is to obtain an initial indication of the analyte characteristic and provide an initial signal corresponding to the initial indication of the analyte characteristic, and
the sensor assembly is to adjust a quantity of the analyte provided to the one or more active test electrodes, per unit time, for said interaction based at least in part on the initial signal.

4. The sensor assembly according to claim 3, wherein the electrode assembly is to adjust the number of active test electrodes of the set of one or more test electrodes.

5. The sensor assembly according to claim 4, wherein:
the electrode assembly is to switch at least one active electrode to an inactive electrode, which inactive electrode does not contribute to the sensor signal, or
the electrode assembly comprises one or more inactive electrodes of the set of electrodes, which one or more inactive electrodes do not contribute to the sensor signal, wherein the sensor assembly is to switch at least one inactive electrode to an active electrode.

6. The sensor assembly according to claim 5, wherein:
the set of one or more test electrodes is spatially distributed about a central axis of symmetry, the applicator assembly being configured such that a path of the sample matrix towards the set of one or more test electrodes is parallel or coaxial with the central axis of symmetry, and
wherein:
the electrode assembly is configured so that switching of at least one active electrode to an inactive electrode comprises switching the active electrode or electrodes located furthest from the central axis to an inactive electrode, and/or
the electrode assembly is configured so that switching of at least one inactive electrode to an active electrode comprises switching the at least one inactive electrode located closest to the central axis to an active electrode.

7. The sensor assembly according to claim 1, wherein the electrode assembly further comprises a set of one or more control electrodes, each control electrode being configured for providing a control measurement which is independent of the analyte, and wherein the electrode assembly is configured so that amounts of analyte provided to each of the set or one or more test electrodes and the set of one or more control electrodes are substantially equal.

8. The sensor assembly according to claim 1, wherein the electrode assembly is to adjust an active electrode surface area thereby adjusting a quantity of the analyte that is delivered to the one or more active test electrodes, per unit time, for said interaction.

9. The sensor assembly according to claim 1, wherein the applicator assembly is to adjust the provision of the sample matrix to the set of one or more test electrodes thereby adjusting a quantity of the analyte that is delivered to the one or more active test electrodes, per unit time, for said interaction.

10. The sensor assembly according to claim 9, wherein the applicator assembly is to adjust the flow rate of the sample matrix and analyte over the set of one or more test electrodes thereby adjusting a quantity of the analyte that is delivered to the one or more active test electrodes, per unit time, for said interaction.

11. A sensor assembly for sensing an analyte in a sample matrix, the sensor assembly comprising:
an electrode assembly comprising a set of one or more test electrodes to interact with the analyte and provide a sensor signal based on said interaction,
wherein the electrode assembly is to define one or more active test electrodes of the set of one or more test electrodes, which one or more active test electrodes contribute to the sensor signal, and
wherein the electrode assembly is to adjust a quantity of the analyte provided to the one or more active test electrodes, per unit time, for said interaction based at least in part on an analyte characteristic by varying an effective active test electrode area.

12. The sensor assembly according to claim 11, wherein varying the effective active test electrode area comprises varying the number of electrodes of the set of one or more test electrodes defined as active electrodes.

13. The sensor assembly according to claim 11, wherein the sensor assembly further comprises an applicator assembly to provide the sample matrix to the set of one or more test electrodes, and wherein the applicator assembly is to adjust the provision of the sample matrix to the set of one or more test electrodes thereby further adjusting a quantity of the analyte that is delivered to the one or more active test electrodes, per unit time, for said interaction.

14. A sensor assembly for sensing an analyte in a sample matrix, the sensor assembly comprising:
an electrode assembly, comprising:
a set of one or more test electrodes, the set of one or more test electrodes comprising an analyte interaction portion to interact with the analyte and provide a sensor signal based on said interaction,
a set of one or more control electrodes, the set of one or more control electrodes providing a control electrode area for providing a control measurement which is independent of the analyte, and
a flow path to provide the sample matrix to the electrode assembly,
wherein the electrode assembly is in the flow path so that amounts of sample matrix provided to each of the set or one or more test electrodes and the set of one or more control electrodes are substantially equal.

15. The sensor assembly according to claim 14, wherein:
the electrode assembly is in the flow path so that the amount of analyte provided to each of the set or one or more test electrodes is 40 to 60% of the total analyte provided to the set of one or more test electrodes and the set of one or more control electrodes combined, and
the electrode assembly is in the flow path so that the amount of analyte provided to each of the set or one or more control electrodes is 40 to 60% of the total analyte provided to the set of one or more test electrodes and the set of one or more control electrodes combined.

16. The sensor assembly according to claim 14, wherein the flow path of the sample matrix towards the electrode assembly defines a central axis, wherein the electrode assembly comprises a substrate with the set of one or more test electrodes and set of one or more control electrodes provided on a first face of the substrate, and wherein the electrode assembly is arranged within the flow path such that the first face of the substrate crosses the central axis of the flow path.

17. The sensor assembly according to claim 16, wherein the first face is substantially perpendicular or perpendicular to the central axis of the flow path.

18. The sensor assembly according to claim 16, wherein the set of one or more test electrodes and the set of one or more control electrodes is spatially distributed about a central axis of symmetry, and wherein the flow path of the sample matrix towards the set of one or more test electrodes is coaxial or parallel with the central axis of symmetry.

19. The sensor assembly according to claim 14, wherein the flow path of the sample matrix towards the electrode assembly defines a central axis, wherein the electrode assembly comprises a substrate with the set of one or more test electrodes and set of one or more control electrodes provided on a first face of the substrate, and wherein the electrode assembly is arranged within the flow path such that flow path is parallel to the first face of the substrate.

20. The sensor assembly according to claim 19, wherein the test electrodes of the set of one or more test electrodes and the control electrodes or the set of one or more control electrodes are interdigitated and arranged in the flow path such that the flow of analyte across the set of one or more test electrodes and the set of one or more control electrodes is substantially equal or equal.
